(19) United States
(12) Patent Application Publication
(10) Pub. No.: US 2021/0304869 A1
(43) Pub. Date: Sep. 30, 2021

(54) Method to Deliver and Use Stress Management Mind-Body Health Tools with Audio Card Technology

(71) Applicant: Root Success Solutions LLC, New London, CT (US)

(72) Inventor: Melissa M. Root, New London, CT (US)

(73) Assignee: Root Success Solutions LLC, New London, CT (US)

(21) Appl. No.: 17/212,972

(22) Filed: Mar. 25, 2021

Related U.S. Application Data
(60) Provisional application No. 62/994,950, filed on Mar. 26, 2020.

(51) Int. Cl. G16H 20/70; G06F 3/16; A61M 21/02

(52) U.S. Cl. CPC G16H 20/70; A61M 2021/0027; A61M 21/02; G06F 3/167

(57) ABSTRACT

A method is described herein comprising obtaining a soundcard comprising an audio device with guidance for at least one of the following: breathing, visualization, and muscle relaxation, and generating an audio output by opening the soundcard. Corresponding systems and devices also are disclosed.

### RELATED APPLICATIONS

This application claims the benefit of U.S. Provisional Patent Application No. 62/994,950 filed Mar. 26, 2020, the contents of which are incorporated by reference in their entirety.

### BACKGROUND

This disclosure relates generally to a method of delivering breathing exercises, guided imagery, and progressive muscle relaxation.

Given the significant escalation of suicide in the United States since 2006, efforts to share effective mental health practices is of paramount importance. However, tools for stress management are historically delivered during in-person sessions with a therapist. Some can also be found on the internet; however, the quality of these online therapies is variable. Apps that contain mind-body health tools exist but use of them is limited to those who seek, download, pay for, and open them.

It would be useful to develop stress-management tools and methods that can be readily accessed by a large number of individuals.

### SUMMARY

One embodiment described herein is a method, comprising obtaining a soundcard comprising an audio device with guidance for at least one of the following: breathing, visualization, and muscle relaxation, and generating an audio output by opening the soundcard.

Another embodiment described herein is a method, comprising embedding an audio device within a soundcard, and triggering a health-related audio output from the audio device when the soundcard is opened.

Yet another embodiment is a soundcard comprising a foldable card with a front section and a back section, and audio device formed in at least one of the front section and the back section that generates a health-related audio output.

### BRIEF DESCRIPTION OF THE DRAWINGS

FIG. 1 shows a perspective view of a first embodiment of a soundcard.

FIG. 2 shows a perspective view of the soundcard in an open position.

FIG. 3A shows back view of the exterior face of the soundcard.

FIG. 3B shows the interior of the soundcard during assembly.

FIG. 4 shows a flow chart of how to generate audio output by opening a soundcard.

FIG. 5 shows a flow chart of how to make a soundcard.

FIG. 6 shows the top view of the first embodiment of a soundcard.

FIG. 7 shows the bottom view of the first embodiment of the soundcard.

FIG. 8 shows the front view of the first embodiment.

FIG. 9 shows the left view of the first embodiment.

FIG. 10 shows the right view of the first embodiment.

FIG. 11 shows the front view of the second embodiment.

FIG. 12 shows the back view of the third embodiment with a central section broken away in a lengthwise direction.

FIG. 13 shows a perspective view of the fourth embodiment.

FIG. 14 shows a perspective view of a fifth embodiment the soundcard with a central section broken away in a widthwise direction.

FIG. 15 shows a perspective view of a sixth embodiment of a soundcard with a central portion broken away in a direction of thickness.

### DETAILED DESCRIPTION

The embodiments disclosed herein include mind-body tools used to address anxiety, depression, and stress management in humans. These tools, when practiced, can allow humans to improve their reactions to stressful situations. Mind-body health tools for stress management are typically utilized by people seeing a therapist, which can be expensive and may have a stigma associated with it, or those with prior knowledge who actively seek them through apps or online searches. There are many others who can benefit from learning positive methods to manage their stress, particularly before they escalate into a mental health crisis. Getting effective mind-body health tools to them in a simple way is a crucial step to address the rapidly escalating mental health challenges evidenced in society. The product and method embodiments described in this application address that gap.

The methods described herein use audio card technology with audio recording of stress management breathing, guided imagery, and progressive muscle relaxation instructions as a method to deliver mind-body health tools. Audio instructions are heard immediately upon opening the audio card. The audio card can be readily handed out by organizations, corporations, schools, and individuals, thus increasing the distribution of mind-body health tools. The connection to the tool is immediate, cost of the audio card is low, need for a therapist for in-person implementation is eliminated, and prior knowledge of mind-body health tools is unnecessary. The methods of delivering mind-body health tools with audio card technology and of using audio card technology to access mind-body health tools are the methods claimed herein. The soundcard can be used without relying on a smartphone or the internet for generating the audio output.

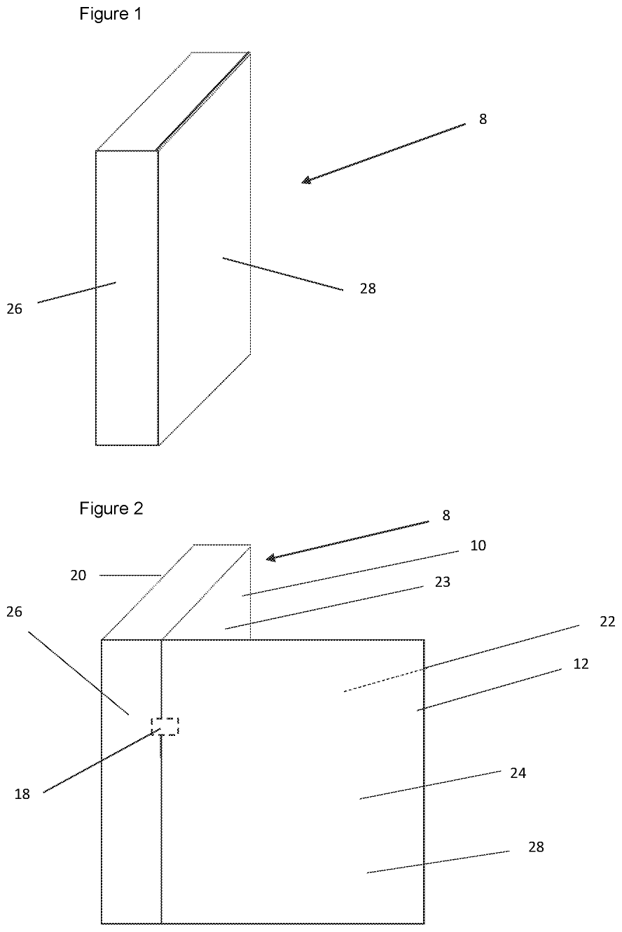

Referring to the drawings, FIG. 1-2, 3A-3B and 6-10 show a first embodiment of the soundcard designated as 8. The soundcard 8 includes a back section 26 and a front section 28, each of which is constructed from two layers of light cardboard or heavy paper. An audio output device 16 is sandwiched between the two layers of cardboard forming the back section 26. The soundcard 8 includes an interior surface 10 that includes, when the soundcard 8 is open, an interior left surface 22, which is part of the front section 28, and an interior right surface 23, which is part of the back section 26. The soundcard 8 also includes an exterior surface 12 that includes an exterior back surface 20 that is part of the back section 26, and an exterior front surface 24 that is part of the front section 28. In the embodiment shown in the figures, the back section 26 is thicker than the front section 28. In other embodiments, the front section 28 can be thicker than the back section 26, or the front section 28 and the back section 26 can have approximately equal thicknesses.

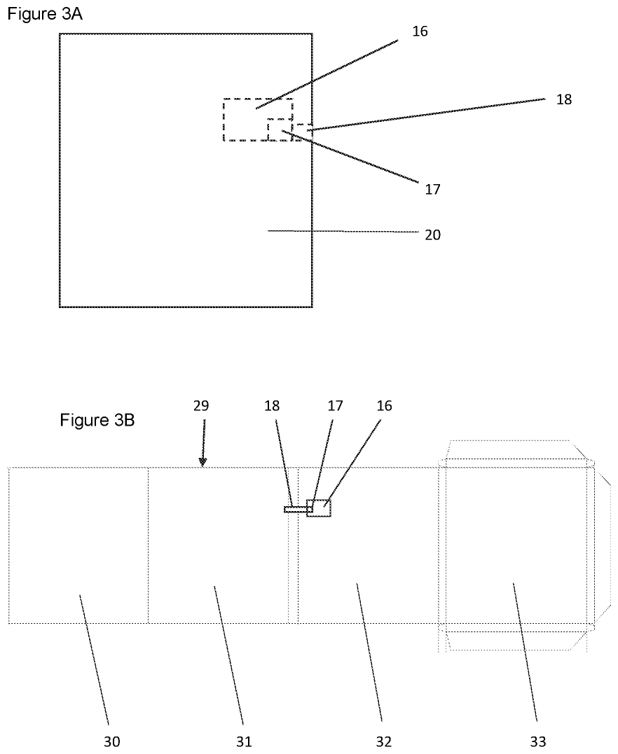

Audio output from the audio output device 16 is initiated when a user opens the soundcard 8 which triggers an activator 18. More specifically, as is shown in FIGS. 3A and 3B, the back section 26 contains an audio output device 16 that contains a battery, a switch 17 and the activator 18. The activator 18 can be a tab that also connects to part of the front section 28 of the soundcard 8, and is pulled by the motion of opening the soundcard 8. When the activator 18 is activated through the motion of opening the soundcard, the switch 17 on the audio output device 16 closes to power the audio output device 16. In other embodiments (not shown), the audio output device 16 can be contained in the front section 28. In embodiments, the activator 18 is a tab that pulls a switched closed when the soundcard 8 is opened.

As is shown in FIG. 3B, the soundcard 8 can be made from a single elongated generally rectangular of cardboard blank 29, which includes first section 30, second section 31, third section 32 and fourth section 33. The first section 30 and the fourth section 33 can be inwardly folded toward the middle. The audio output device can be sandwiched between the two layers formed by sections 32 and 33. The perimeters of the folded (and open) card can be connected with an adhesive. When the card is opened, a switch is closed to connect a battery power source to the audio output device in order to play the recorded sound. In some cases, the switch 17 is closed by a tab that is connected to the interior surface 22 of the front on the card, and this tab pulls the switch closed when the soundcard 8 is opened.

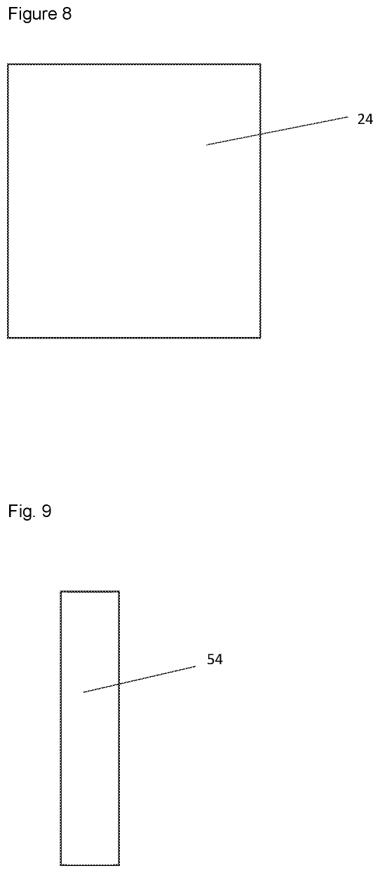

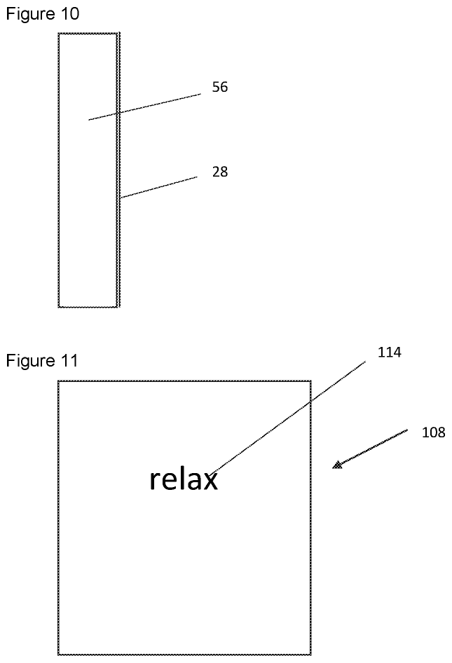

Additional views of the soundcard are included. FIG. 6 shows the top surface 50 of the back section 26. FIG. 7 shows the bottom surface 52 of the bottom surface of the back section 26. FIG. 8 shows the front of the soundcard 8. FIG. 9 shows the left side surface of the soundcard 8, and FIG. 10 shows the right side surface of the front section 28.

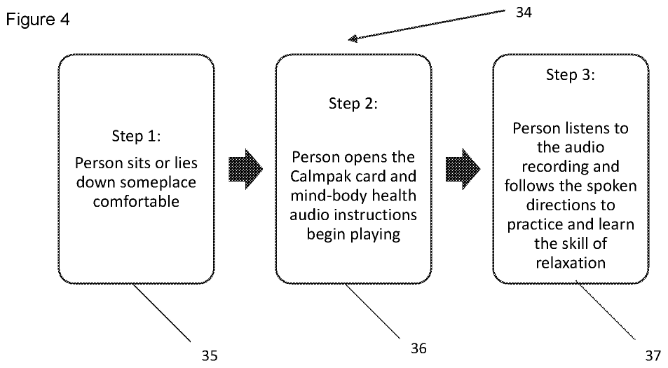

FIG. 4 shows a flow chart of how to generate audio output by opening a soundcard. The overall method is indicated as 34. In step 1 at 35, the user sits or lies down in a comfortable place. In step 2 at 36, the user opens the soundcard and the mind-body health audio instructions begins playing. In step 3 at 37, the user listens to the audio recording and follows the spoken directions to practice and learn the skill of relaxation.

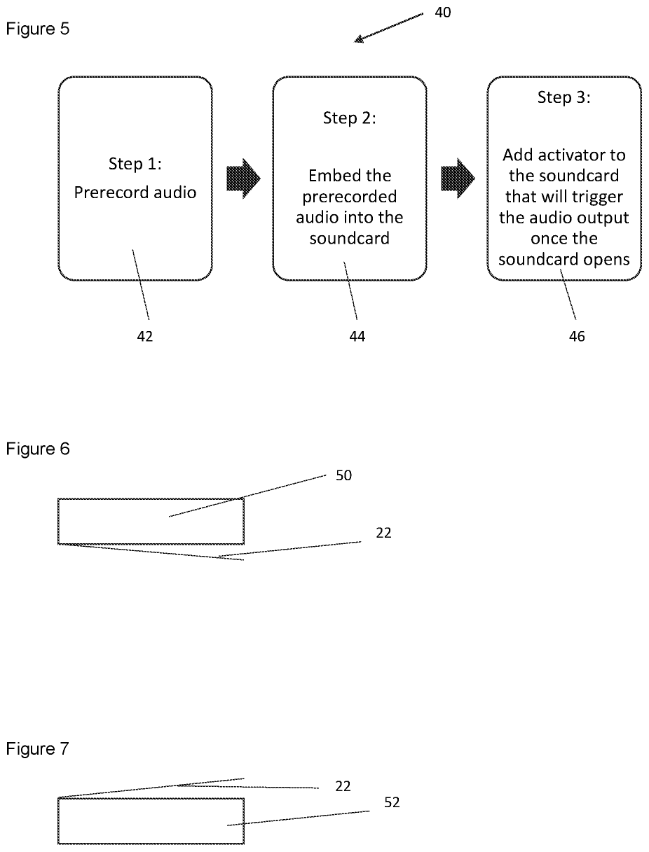

FIG. 5 shows a flow chart of how to make a soundcard. The overall process is designated at 40. In step 1 at 42, the audio is pre-recorded onto the soundcard. In step 2 at 44, the pre-recorded audio is embedded into the soundcard. In step 3 at 46, the audio output device is connected to an activator that will cause the audio output to play when the soundcard is opened. After recording, the soundcard can be transferred to a user, or to another user.

FIG. 11 shows a second embodiment of the soundcard designated as 108. The word “relax” is included on the front, as shown at 114, in order to remind the user the practice and/or use relaxation techniques with the aid of the audio output.

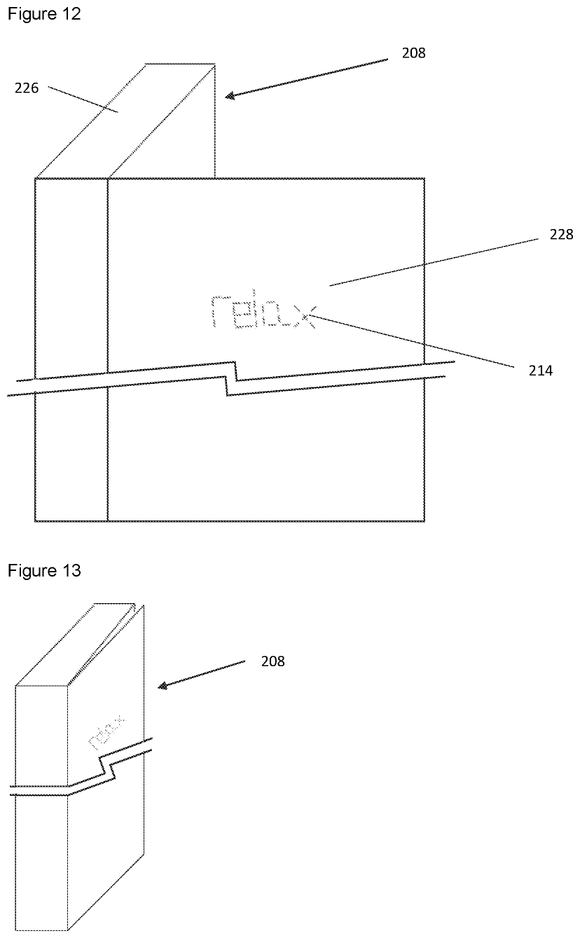

FIGS. 12 and 13 show a third embodiment of the soundcard designated as 208, which has a front section 228 and a back section 226. The word “relax” 214 is optional. The length of the soundcard 208 can be varied.

FIG. 13 shows a fourth embodiment of the soundcard designated as 308, which has a front section 328 and a back section 326. The figure is broken away at the back section 326 to show that the soundcard 308 can have various thicknesses.

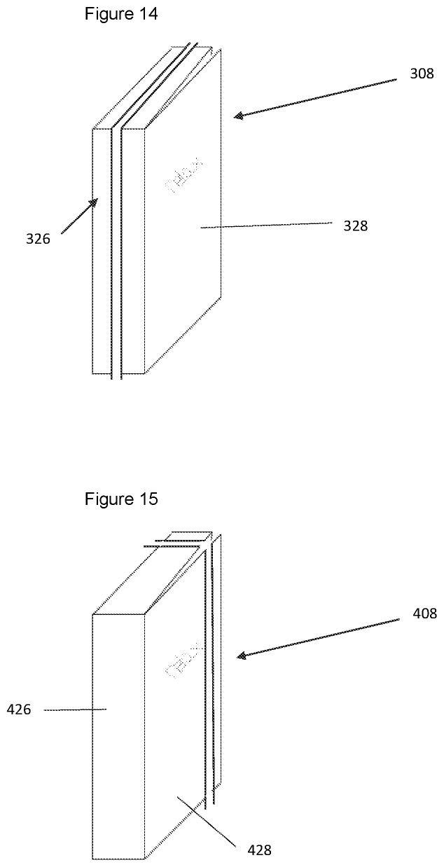

FIG. 14 shows a fifth embodiment of the soundcard designated as 408, which has a front section 428 and a back section 426. The figure is broken away to show that the soundcard 408 can have various widths.

In most cases, the sound emitted from the audio output device includes music and/or speech. In embodiments, the assembled soundcard has a vertical length of about 100 mm to 150 mm, or about 110 mm to about 140 mm, or about 120 mm to about 135 mm. The back has a thickness in the range of about 5 mm to about 10 mm, or about 6 mm to about 9 mm. The front has a thickness of about 0.5 mm to about 2 mm, or about 0.5 mm to about 1 mm.

A number of alternatives, modifications, variations, or improvements therein may be subsequently made by those skilled in the art, which are also intended to be encompassed by the following claims. The claims cover more than what is shown in drawings.

What is claimed is:

1. A method, comprising:
obtaining a soundcard comprising an audio device with guidance for at least one of the following: breathing, visualization, and muscle relaxation, and
generating an audio output by opening the soundcard.

2. The method of claim 1, wherein the soundcard's presence reminds a user to practice stress management techniques.

3. The method of claim 1, wherein the soundcard is configured to allow a user to listen to the audio output and follow spoken directions to practice and learn the skill of relaxation.

4. The method of claim 1, wherein the soundcard is dimensioned to be capable of being held in a hand of a user.

5. The method of claim 1, further comprising transferring the soundcard to another user.

6. The method of claim 1, wherein the soundcard can be used without relying on a smartphone or the internet for generating the audio output.

7. The method of claim 1, wherein the audio output comprises pre-recorded audio.

8. The method of claim 1, further comprising a user practicing the techniques spoken by the audio output in the soundcard.

9. The method of claim 1, wherein a user sits or lies down someplace comfortable prior to opening the soundcard.

10. The method of claim 1, further comprising stopping the audio output when the soundcard is closed.

11. A method, comprising:
embedding an audio device within a soundcard, and
triggering a health-related audio output from the audio device when the soundcard is opened.

12. The method of claim 11, wherein the soundcard has the term “relax” displayed on a surface.

13. The method of claim 11, wherein an activator triggers the audio output.

14. The method of claim 13, wherein the activator closes a switch that powers the audio device to generate the audio output.

15. The method of claim 11, wherein the health-related audio output comprises stress-reduction guidance for at least one of breathing, visualization, and muscle relaxation.

16. A soundcard, comprising:
a foldable card with a front section and a back section, and
audio device formed in at least one of the front section and the back section that generates a health-related audio output.

17. The soundcard of claim 16, wherein the soundcard is dimensioned to be capable of being held in a hand of a user.

18. The soundcard of claim 16, wherein an activator triggers the audio output.

19. The soundcard of claim 16, wherein the activator closes a switch when the soundcard is opened.

20. The soundcard of claim 16, wherein, after assembly, the soundcard is a one-fold card with the back section being thicker than the front section.
